(19) United States
(12) Patent Application Publication
(10) Pub. No.: US 2007/0160484 A1
(43) Pub. Date: Jul. 12, 2007

(54) BOOSTER-TYPE GAS COMPRESSOR

(76) Inventor: Hiroshi Inoue, Yokohama-shi, Kanagawa (JP)

Correspondence Address: OSTROLENK FABER GERB & SOFFEN, 1180 AVENUE OF THE AMERICAS, NEW YORK, NY, 100368403, US

(21) Appl. No.: 11/621,721

(22) Filed: Jan. 10, 2007

(30) Foreign Application Priority Data
Jan. 10, 2006 (JP) 2006-2210

(51) Int. Cl. A61M 1/00

(52) U.S. Cl. 417/437

(57) ABSTRACT

A booster-type gas compressor comprises a compressed-gas flow path, a cylinder, and a piston that moves up and down in the cylinder to further compress a compressed gas fed into the cylinder from the compressed-gas flow path. In a crank case, a crank shaft is rotated with a driving shaft to move the piston up and down. The compressed-gas flow path is connected to the crank case via a bypath conduit to make pressure difference above and under the piston reduced.

### BACKGROUND OF THE INVENTION

The present invention relates to a booster-type gas compressor in which a compressed gas is compressed by a reciprocating piston.

As shown in FIG. 4, a booster-type gas compressor is known, in which a suction valve 23 and a discharge valve 24 are provided at one end and the other end respectively of a top wall 22 of a cylinder 21. Over the suction valve 23 and discharge valve 24, a suction chamber 26 having an inlet 25 and a discharge chamber 28 having an outlet 27 are provided. By reciprocating a piston 33 in a cylinder 21 with a piston rod 32 by a crank shaft 31 of a driving shaft 30 driven by an external power source, a compressed gas such as N₂ gas sucked from a compressed-gas flow path 34 through the inlet 25 is introduced into a chamber on the piston 33, further compressed and discharged through the discharge valve 24 and outlet 27.

The driving shaft 30 is rotatably secured through one side wall 29a of the crank case 29 via a seal 35 and a ball bearing 36, and one end of the driving shaft 30 is rotatably secured on the other side wall 29b of the crank case 29 via a ball bearing 37.

One end of the piston rod 32 is rotatably secured to a crank shaft 31 of the driving shaft 30 via a ball bearing 38. In the crank case 29, there is an air hole 39 which communicates with external air.

In a known booster-type gas compressor as shown in FIG. 4, with reciprocating motion of the piston 33, a compressed gas such as N₂ gas which flows into the chamber on the piston 33 in the cylinder 21 leaks through around the piston 33 to come in the crank case 29 having low pressure.

The compressed gas which flows into the crank case 29 is discharged through the air hole 39 of the crank case 29 to air which causes energy loss. A toxic gas causes air pollution.

During compression step of the piston 33, internal pressure in the compressing chamber becomes greater, while the inside of the crank case 29 under the piston 33 is substantially equal to atmospheric pressure. Thus, pressure difference gives the piston 33 rapid high force, so that excessive and unequal force is added not only to the outer circumferential surface of the piston 33 and piston ring but also to a pivot portion and a sealing portion.

Excessive force which changes direction continually acts to each sliding portion and rotating portion to make wear and damage earlier to cause poor performance for a long time.

### SUMMARY OF THE INVENTION

In view of the disadvantages in the prior art, it is an object of the invention to provide a booster-type gas compressor in which difference in pressure above and under a piston is reduced to prevent unequal load to parts.

### BRIEF DESCRIPTION OF THE DRAWINGS

The features and advantages of the invention will become more apparent from the following description with respect to embodiments as shown in accompanying drawings wherein:

FIG. 1 is a vertical sectional view showing the first embodiment of a booster-type gas compressor according to the present invention;

FIG. 2 is a vertical sectional view showing the second embodiment of a booster-type gas compressor according to the present invention;

FIG. 3 is a vertical sectional view showing the third embodiment of a booster-type gas compressor according to the present invention; and

FIG. 4 is a vertical sectional view of a known booster-type gas compressor.

### DETAILED DESCRIPTION OF PREFERRED EMBODIMENTS

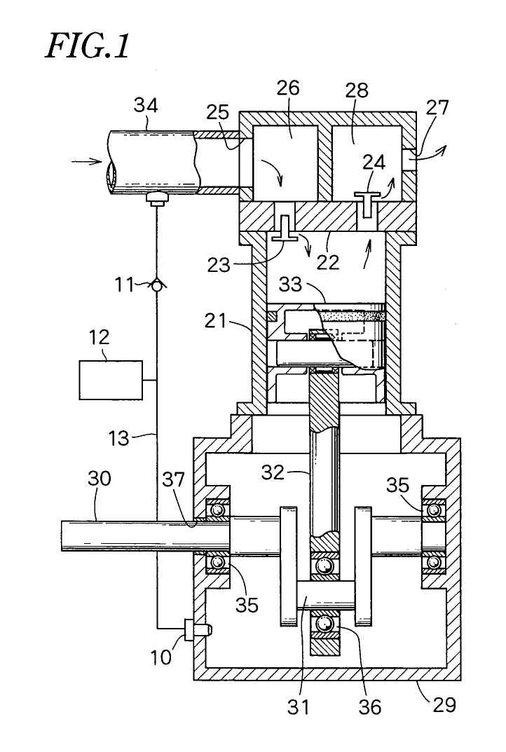

FIG. 1 shows the first embodiment of a booster-type gas compressor according to the present invention.

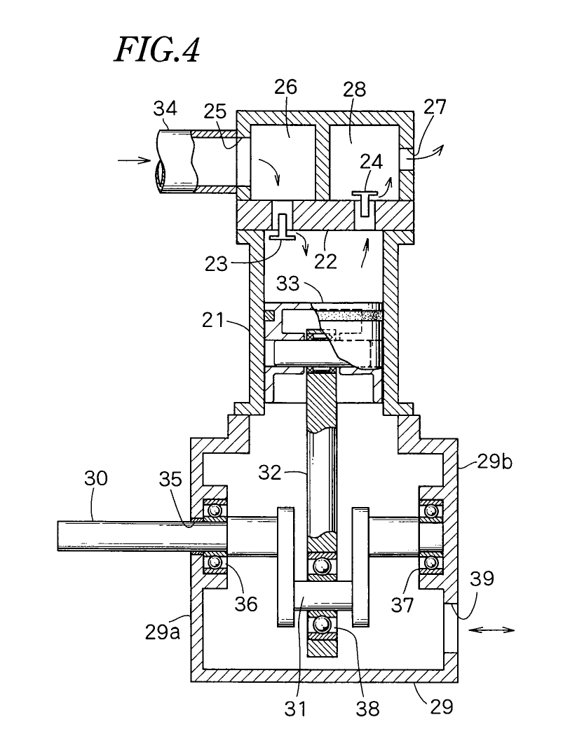

The basic structure of the booster-type gas compressor is not so different from that in FIG. 4. The same numerals are allotted to the same members as those in FIG. 4 and description thereof is omitted. Only difference will be described.

In FIG. 1, there is no air hole 39 of FIG. 4. Instead, a compressed-gas inlet 10 is formed. A compressed-gas flow path 34 is connected to the compressed-gas inlet 10 via a bypath conduit 13 comprising a check valve 11 which closes towards the compressed-gas flow path 34 and opens in an opposite direction and a pressure-regulator 12 such as a pressure-regulating valve or a pressure reducing valve.

When a compressed gas is fed to an air-suction chamber 26 by opening a valve (not shown), it is partially fed to a crank case 29 via the bypath conduit 13 comprising the check valve 11 and the pressure regulator 12 and the compressed-gas inlet 10, so that the inside of the crank case 29 is compressed to more than atmospheric pressure.

The compressed gas in the compressed-gas flow path 34 is fed into the crank case 29 in which pressure of the gas becomes more than atmospheric pressure different from a conventional device.

The difference in pressure of the gas between a compressing chamber above a piston of a cylinder 21 and the crank case becomes smaller than that in a conventional device, so that the piston 3 becomes unlikely to lack smooth sliding, or ball bearings 36-38 and a seal 35 becomes unlikely to reduce their lives or to produce play between them.

By the pressure regulator 12, pressure of the compressed gas in the crank case 29 is regulated, so that difference in pressure to the compressing chamber above the piston is regulated as soon as possible thereby achieving stable performance.

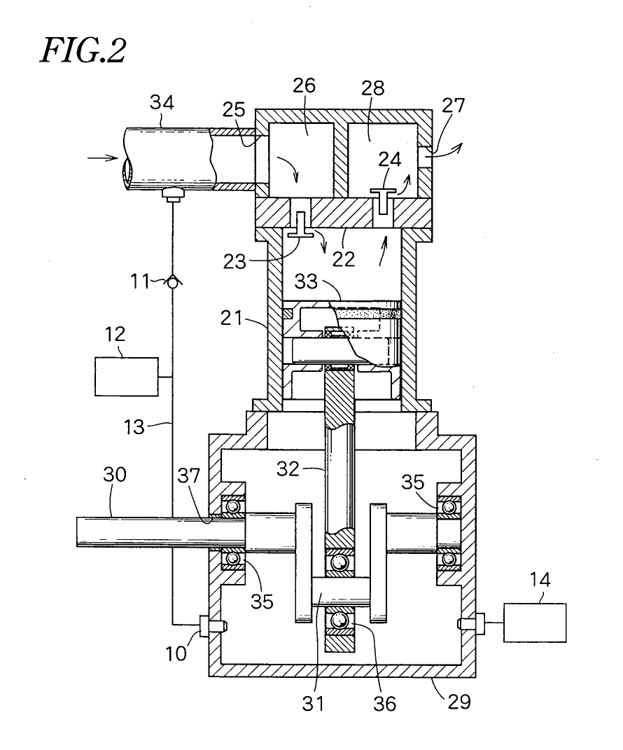

In the second embodiment of the present invention in FIG. 2, a pressure regulator 14 is joined to a crank case 29. The pressure regulator 14 comprises a reserve tank comprising a pressure-regulating valve and a check valve.

Similar advantages to FIG. 1 are achieved.

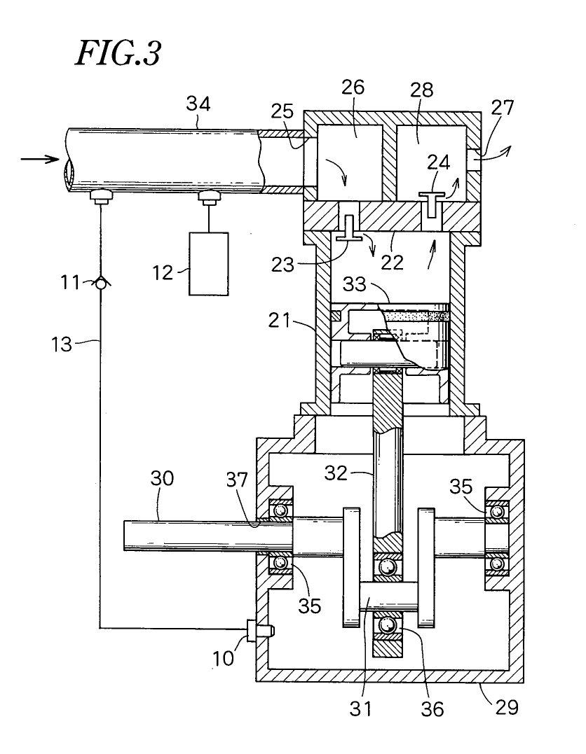

FIG. 3 shows the third embodiment of the present invention, in which a compressed-gas flow path 34 is connected to a compressed-gas inlet 10 of a crank case 29 via a bypath conduit 13 comprising a check valve 11 which closes towards the compressed-gas flow path 34 and opens in an opposite direction. A pressure regulator 12 is provided at the compressed-gas flow path 34 between the bypath conduit 13 and a suction valve 23.

The foregoing merely relates to embodiments of the invention. Various changes and modifications may be made by a person skilled in the art without departing from the scope of claims wherein:

What is claimed is:

1. A booster-type gas compressor comprising:
a compressed-gas flow path;
a cylinder into which a compressed gas is introduced from the compressed-gas flow path;
a piston that moves up and down in the cylinder to further compress the compressed gas in the cylinder;
a piston rod coupled to the piston at an upper end;
a crank shaft coupled to a lower end of the piston rod;
a driving shaft coupled to the crank shaft to rotate with the crank shaft to allow the piston up and down;
a crank case including the crank shaft under the piston; and
a bypath conduit that connects the compressed-gas flow path to the crank case.

2. A booster-type gas compressor of claim 1 further comprising a pressure regulator on the way of the bypath conduit.

3. A booster-type gas compressor of claim 1 further comprising a pressure regulator coupled to the crank case.

4. A booster-type gas compressor of claim 1 further comprising a pressure regulator coupled to the compressed-gas flow path.

5. A booster-type gas compressor of 1 further comprises a check valve on the way of the bypath conduit that makes the compressed gas flow only towards the crank case.
